(19) United States
(12) Patent Application Publication
(10) Pub. No.: US 2013/0210030 A1
(43) Pub. Date: Aug. 15, 2013

(54) IN VIVO METHOD FOR THE EVALUATION OF A COMPOUND-TARGET INTERACTION

(71) Applicants: Gerard Drewes, Heidelberg (DE); Carsten Hopf, Mannheim (DE); Gitte Neubauer, Mannheim (DE); Ulrich Kruse, Dossenheim (DE)

(72) Inventors: Gerard Drewes, Heidelberg (DE); Carsten Hopf, Mannheim (DE); Gitte Neubauer, Mannheim (DE); Ulrich Kruse, Dossenheim (DE)

(73) Assignee: Cellzome AG, Heidelberg (DE)

(21) Appl. No.: 13/809,811

(22) PCT Filed: Jun. 20, 2011
(86) PCT No.: PCT/EP2011/003055

§ 371 Date: Apr. 13, 2013

(30) Foreign Application Priority Data
Jul. 19, 2010 (EP) 10007451.7

(51) Int. Cl. G01N 33/573

(52) U.S. Cl. CPC G01N 33/573
USPC 435/7.4

(57) ABSTRACT

The invention relates to a method for evaluation of a compound-target interaction in vivo, comprising the steps of administering the compound to an animal, taking a body fluid sample of the animal, determining the concentration of the compound in the body fluid sample, taking a cellular sample of the animal containing the target, preparing a protein preparation of said cellular sample, providing an immobilised ligand capable of binding to the target, contacting the protein preparation with the immobilised ligand under conditions allowing the formation of a complex between the immobilised ligand and the target, determining the amount of complexes formed in step, and correlating the amount of complexes with the concentration of the compound in the body fluid sample.

The present application relates to an in vivo method for the evaluation of a compound-target interation wherein the compound is adminstered to an animal and wherein the concentration of the compound in a body fluid sample is correlated with the amount of complexes formed between the target and the compound in a cellular sample of the animal.

In drug discovery it is desirable to know which percentage of a drug target needs to be occupied by a drug in order to elicit a therapeutic effect in animal models and patients. Measurement of target occupancy, also referred to as target engagement, can guide dosing of compounds in preclinical and clinical drug development. This information can be especially useful to establish initial dosing recommendations for new drug targets where little information is available on their functional responses (Grimwood and Hartig, 2009. Pharmacol. Ther. 122(3):281-301).

Measurement of target occupancy can guide optimal dosing of the compound of interest, for example a drug candidate, to achieve specified levels of target occupancy which effectively inhibit the target of interest, for example an enzyme, with minimal side effects. This dose response relationship can be used to predict clinical efficacy for novel compounds. In addition, time-dependent target occupancy data can provide information on the onset, steady state and duration of drug effects.

Information about target occupancy is useful in the early stages of drug discovery to select a suitable compound for further development but also at later stages, for example dose selection for clinical trials. For example, initial dose-ranging studies in clinical trials are often based on large patient groups using symptom evaluation as primary response. These early, dose-ranging patient groups may be smaller and timelines shortened if instead of primary outcome the necessary target occupancy level determined in a preclinical model is measured. Furthermore, many clinical trials showed no evidence for efficacy, but it was not known whether a sufficient level of drug reached the intended target site or stayed long enough on the target (Grimwood and Hartig, 2009. Pharmacol. Ther. 122(3):281-301).

Several different methods are known to obtain target occupancy data. A standard method is based on in vivo radioligand binding. The in vivo interaction of the test compound and tracer amounts of a radioligand is used to estimate target occupancy of drug targets, for example G protein-coupled receptors. Typically these studies use rodents injected with [³H]-labeled radioligands (Hirst et al., 2008. J. Pharmacol. Exp. Ther. 325(1):134-145).

Another method involves ex vivo radioligand binding with in vivo equilibration of test compound followed by an in vitro assay of residual sites that are not occupied by the test compound in vivo (Stean et al., 2002. Pharmacol. Biochem. Behav. 71(4):645-54). Both in vivo and ex vivo binding studies are routinely performed with organs that can be easily dissected. Autoradiographic readouts can be used for smaller regions, for example specific brain regions.

Micro Positron Emission Tomography (microPET) is a method that can be directly translated to clinical studies because tracers used for animal studies can often times be also used for human PET studies. This technique uses tomographic imaging of a living animal with 1-2 mm resolution using a gamma camera (Single Photon Emission Computing Tomography, SPECT) or ring of coincidence detectors (PET). A significant challenge is that the animal needs to be immobilized during scanning, for example by sedation which can influence the animal's normal physiology (Riemann et al, 2008. Q J Nucl. Med. Mol. Imaging. 52(3):215-221).

PET ligands have also been developed for the imaging of enzymes (Gatley et al., 2003. Drug Dev. Res. 59, 194-207). For example, [¹¹C]Clorgyline and [¹¹C]L-deprenyl are selective mechanism-based irreversible inhibitor tracers for monoamine oxidase A (MAO-A) and the glial enzyme monoamine oxidase B (MAO-B), respectively (Fowler et al., 2005. Mol. Imaging. Biol. 7(6):377-387).

Another method was reported for correlating drug exposure, target occupancy and efficacy for protein therapeutics such as antibodies. This in vivo approach was used to determine the level of blockade required to inhibit the generation of a T cell-dependant antibody response (Metz et al., 2009. Eur. J. Pharmacol. 610(1-3):110-118).

It is the object of the present invention to provide a method for the in vivo evaluation of compound-target interactions.

Accordingly, the present invention provides a method for evaluation of a compound-target interaction in vivo, comprising the steps of
a) administering the compound to an animal,
b) taking a body fluid sample of the animal,
c) determining the concentration of the compound in the body fluid sample,
d) taking a cellular sample of the animal containing the target,
e) preparing a protein preparation of said cellular sample,
f) providing an immobilized ligand capable of binding to the target,
g) contacting the protein preparation with the immobilized ligand under conditions allowing the formation of a complex between the immobilized ligand and the target,
h) determining the amount of complexes formed in step g), and
i) correlating the amount of complexes with the concentration of the compound in the body fluid sample.

As demonstrated in the examples, with the method of the present invention, it is possible to accurately determine the interaction of a compound with its target in vivo. Especially, it is possible to determine a dose response relationship correlating the concentration of the compound in plasma to the degree of target binding.

Consequently, the present invention provides an in vivo pharmacodynamic method for studying compound-target interactions.

The present invention is based on the concept that the compound to be tested is administered to an animal and then absorbed by the animal into at least some of the cells containing the potential target of the compound. Then, a protein preparation of at least some of these cells is taken. This protein preparation is then contacted with a known, immobilized ligand of the target. The more compounds have interacted with the target, the less complexes between the target and the ligand are formed. Consequently, the amount of the complexes between the ligand and the target is reciprocally linked to the amount of the compound that entered the cell and bound to the target. By correlating the amount of complexes with the concentration of the compound in a body fluid of the animal, it is therefore possible to correlate the compound concentration in vivo and its interaction with the target. The doses which prove to be optimal in the method of the present invention as determined in a preclinical animal model may be used in a clinical trial design protocol and particularly in a human clinical design protocol.

According to the first step of the method of the present invention, the compound to be tested is administered to an animal.

According to one aspect of the present invention, the claimed method contains steps performed on the animal body. However, these steps do not require the action of veterinarian or of a medical doctor.

In the context of the present invention, it is possible to test any compound which is capable of interacting with a cellular target. Preferably, the target is an intracellular target. Equally preferred is that the target is a membrane protein, for example a receptor protein such as a receptor tyrosine kinase, for example a member of the EGF receptor family. Equally preferred is a protein that is localized on the cell surface.

In the context of the present invention, it is also possible to test any compound which is capable of interacting with an extracellular target, for example a growth factor, cytokine or chemokine that is secreted by cells and circulates, for example, in the blood (e.g. tumor necrosis factor α (TNFα) and vascular endothelial growth factor (VEGF)).

Preferably, the target is the target that should be modulated by the drug in order to achieve its pharmacological and therapeutic effect.

Equally preferred is the measurement of the interaction of the compound with so-called off targets which can mediate unwanted side effects. For example, a non-selective kinase inhibitor may bind also to several other kinases than the therapeutic target kinase. Information of the occupancy of off-targets at a given compound concentration is expected to give valuable guidance for dose selection. For example, a therapeutic antibody may cross-react with other antigens in addition to the intended antigen against which the antibody was raised.

Preferably, said compound is selected from the group consisting of synthetic or naturally occurring chemical compounds or organic synthetic drugs, more preferably small molecule organic drugs or natural small molecule compounds. Such small molecules are preferably not proteins or nucleic acids. Preferably, small molecules exhibit a molecular weight of less than 1000 Da, more preferred less than 750 Da, most preferred less than 500 Da. It is also possible said compound is identified starting from a library containing such compounds. Then, in the course of the present invention, a collection of such molecules or a library of molecules is screened.

A “library” according to the present invention relates to a collection of different chemical entities that are provided in a sorted manner that enables both a fast functional analysis (screening) of the different individual entities, and at the same time provide for a rapid identification of the individual entities that form the library. Libraries of synthetic and natural origins can either be purchased or designed by the skilled artisan. In an alternatively preferred embodiment, said compound is an antibody.

In the context of the present invention, the term “antibody” refers to any kind of immunoglobulin-derived structure with binding specificity to an antigen, including, but not limited to, a full length antibody, an antibody fragment (a fragment derived, physically or conceptually, from an antibody structure), a derivative of any of the foregoing, a chimeric molecule, a fusion of any of the foregoing with another polypeptide, or any alternative structure/composition. An antibody of the invention may be any polypeptide which comprises at least one antigen binding fragment. Antigen binding fragments consist of at least the variable domain of the heavy chain and the variable domain of the light chain, arranged in a manner that both domains together are able to bind to the specific antigen. An antibody fragment contains at least one antigen binding fragment as defined above, and exhibits essentially the same function and specificity as the complete antibody of which the fragment is derived from.

Preferably, the antibody of the invention is selected from the group consisting of monoclonal antibodies, polyclonal antibodies, chimeric antibodies, and antibody fragments.

Monoclonal antibodies are monospecific antibodies that are identical because they are produced by one type of immune cell that are all clones of a single parent cell. Polyclonal antibodies include, for example, antibodies derived from a patient suffering from an autoimmune disease. A chimeric antibody is an antibody in which at least one region of an immunoglobulin of one species is fused to another region of an immunoglobulin of another species by genetic engineering in order to reduce its immunogenecity. For example, murine V_{L} and V_{H} regions may be fused to the remaining part of a human immunoglobulin. In addition, the antibody of the present invention may be an antibody fragment in form of an antibody domain (Fab), a single chain antibody, or a biological receptor or receptor fusion protein (Chames et al., 2009. Br. J. Pharmacol. 157(2): 220-233).

Examples of therapeutic antibodies include Panitumumab (Vectibix®) and Cetuximab (Erbitux®) which both target the human EGF receptor as well as Trastuzumab (Herceptin®) which targets human epidermal growth factor receptor-2 (HER2; Jiang et al., 2011. Nat. Rev. Drug. Discov. 10(2): 101-111).

Furthermore, the compound may have already been tested in vitro for its capacity of interacting with the target. Then, the method of the present invention provides a suitable possibility of further evaluating this compound in vivo.

The compound may be a modulator, preferably an inhibitor of the target.

Preferably, the inhibitor is a reversible inhibitor that binds non-covalently to the target.

Equally preferred, the inhibitor is an irreversible inhibitor that binds covalently to the target.

The scope of targets that can be inhibited by irreversible inhibitors is broad, including enzymes such as kinases and proteases but also non-enzyme proteins such as transthyretin (TTR). Examples of irreversible kinase inhibitors are inhibitors of the EGF receptor (e.g. Neratinib) and Brutons Tyrosine kinase (BTK) (e.g. AVL-292 and PCI-32765) (Singh et al., 2011. Nat. Rev. Drug Discov. 10(4):307-317).

The methods of the present invention are especially useful to study the pharmacodynamic effects of irreversible inhibitors. Irreversible inhibitors may have advantages compared to reversible inhibitors because they may display a different pharmaco-dynamic relationship. After irreversible inhibition of the target, a re-synthesis of the protein may be necessary to restore its function. Therefore, the prolonged duration of the drug action may uncouple the pharmacodynamics of the drug from the pharmacokinetic exposure (Singh et al., 2011. Nat. Rev. Drug Discov. 10(4): 307-317).

The compound may be administered to the animal in any suitable way, including but not limited to oral and parenteral administration, for example intravenous, subcutaneous, intraperitoneal, or intracerebral administration.

Preferably, the compound is administered orally.

In principle, each animal can be used in accordance with the present invention, as long as the target of the compound is present in said animal. Methods for determining whether the target is present are known in the art and include e.g. in situ hybridization, RT-PCR, Western Blotting, or proteomics analysis.

The animal may be a mammal including humans. Preferably, the animal is a non-human animal, e.g. a mammal, e.g. a rabbit or dog or a rodent including a mouse or a rat, or a primate including a monkey, e.g. a rhesus monkey or a cynomolgus monkey.

According to the invention, the term “target” refers to an entity occurring in the animal which can be targeted, preferably bound by the compound. Preferably, the target is a protein, preferably an enzyme which is preferably a kinase.

In an alternatively preferred embodiment, the target is an antigen.

In the context of the present invention, the term “antigen” refers to any biomolecule to which an antibody can bind, including, but not limited to, peptides, proteins, lipids and nucleic acids.

In a further step of the method of the present invention, a body fluid sample of the animal is taken.

According to the present invention, the term “body fluid” refers to any liquid in the body derived e.g. from blood, plasma, lymph liquid, or cerebrospinal fluid. Methods for taking these body samples are generally known in the art and, as the skilled artisan will appreciate, depend both from the animal used as well as from the nature of the body sample.

However, it is also included within the present invention that instead of a body fluid sample, a cellular sample derived e.g. from an organ of the animal like the brain is taken for the measurement of the concentration of the compound.

In a further step, the concentration of the compound in the body fluid sample is determined. Such methods are known in the art and will depend on the chemical nature of the compound. Suitable methods include mass spectrometry and HPLC-MS/MS methods

In the context of the present invention, it is equally possible that instead of steps a) to c) of the claimed method, a step of determining the concentration of the compound in a body fluid sample of an animal which has been administered with the compound is performed. For this step, all embodiments described above with respect to steps a) to c) also apply.

In a further step of the method of the present invention, a cellular sample of the animal containing the enzyme is taken.

The animal may be sacrificed before steps b) and/or d) are performed.

According to the present invention, the term “cellular sample” relates to any sample taken from the animal which contains at least one cell, provided that said cell or cells contain the target. Preferably, but not limited to these embodiments, the cellular sample is derived from the blood, an organ or a tumor tissue, including a xenograft tumor, of the animal. The sample can be taken from the animal e.g. by biopsy or after sacrifying the animal. Corresponding methods are known in the art. For example, a biopsy is a diagnostic procedure used to obtain a small amount of tissue, which can then be examined microscopically or with biochemical methods. Biopsies are important to diagnose, classify and stage a disease, but also to evaluate and monitor drug treatment.

In detail, the choice of the cell will mainly depend on the expression of the target, since it has to be ensured that the target is principally present in the cell of choice. In order to determine whether a given cell is a suitable starting system for the methods of the invention, methods like Westernblot, PCR-based nucleic acids detection methods, Northernblots and DNA-microarray methods (“DNA chips”) might be suitable in order to determine whether a given target of interest is present in the cell.

The choice of the cell may also be influenced by the purpose of the study. If the in vivo efficacy for a given drug needs to be analyzed then cells or tissues may be selected in which the desired therapeutic effect occurs (e.g. T-cells). By contrast, for the elucidation of protein targets mediating unwanted side effects, the cell or tissue may be analysed in which the side effect is observed (e.g. cardiomyocytes).

In a further step of the present invention, a protein preparation of said cellular sample is prepared. Examples of a protein preparation include a cell lysate or a partial cell lysate which contains not all proteins present in the original cell, as long as it contains the target.

Methods for the lysis of cells are known in the art (Karwa and Mitra: Sample preparation for the extraction, isolation, and purification of Nuclei Acids; chapter 8 in “Sample Preparation Techniques in Analytical Chemistry”, Wiley 2003, Editor: Somenath Mitra, print ISBN: 0471328456; online ISBN: 0471457817). Lysis of different cell types and tissues can be achieved by homogenizers (e.g. Potter-homogenizer), ultrasonic desintegrators, enzymatic lysis, detergents (e.g. NP-40, Triton X-100, CHAPS, SDS), osmotic shock, repeated freezing and thawing, or a combination of these methods.

Partial cell lysates can e.g. be obtained by isolating cell organelles (e.g. nucleus, mitochondria, ribosomes, golgi etc.) first and then preparing protein preparations derived from these organelles. Methods for the isolation of cell organelles are known in the art (Chapter 4.2 Purification of Organelles from Mammalian Cells in “Current Protocols in Protein Science”, Editors: John. E. Coligan, Ben M. Dunn, Hidde L. Ploegh, David W. Speicher, Paul T. Wingfield; Wiley, ISBN: 0-471-14098-8).

In addition, protein preparations can be prepared by fractionation of cell extracts thereby enriching specific types of proteins such as cytoplasmic or membrane proteins (Chapter 4.3 Subcellular Fractionation of Tissue Culture Cells in “Current Protocols in Protein Science”, Editors: John. E. Coligan, Ben M. Dunn, Hidde L. Ploegh, David W. Speicher, Paul T. Wingfield; Wiley, ISBN: 0-471-14098-8).

In a preferred embodiment, cells isolated from peripheral blood represent a suitable biological material. Procedures for the preparation and culture of human lymphocytes and lymphocyte subpopulations obtained from peripheral blood (PBLs) are widely known (W. E Biddison, Chapter 2.2 “Preparation and culture of human lymphocytes” in Current Protocols in Cell Biology, 1998, John Wiley & Sons, Inc.). For example, density gradient centrifugation is a method for the separation of lymphocytes from other blood cell populations (e.g. erythrocytes and granulocytes). Human lymphocyte subpopulations can be isolated via their specific cell surface receptors which can be recognized by monoclonal antibodies. The physical separation method involves coupling of these antibody reagents to magnetic beads which allow the enrichment of cells that are bound by these antibodies (positive selection).

In the context of the present invention, it is equally possible that instead of steps d) and e) of the present invention, a step of preparing a protein preparation of a cellular sample obtained from the animal of step a) is performed. For this step, all embodiments described above with respect to steps d) and e) also apply.

Furthermore, according to the present invention, an immobilized ligand capable of binding to the target is provided.

According to the present invention, the term ligand relates to any compound known to bind the target. Preferably, said compound is as defined above. For example, if the target is an enzyme like a kinase, a compound known to bind the enzyme can be taken as the ligand. Typically the ligand binds to the same protein pocket to which the drug is directed, for example the ATP-binding pocket of kinases.

Preferably, said ligand is an antibody.

In another preferred embodiment, the compound and the ligand of the invention are both antibodies.

According to the invention, said ligand is immobilized. As used herein, the term “immobilized” means that the ligand is bound, preferably covelantly bound to a solid support. The term “solid support” relates to every undissolved support being able to immobilize the compound on its surface. The solid support may be selected from the group consisting of agarose, modified agarose, sepharose beads (e.g. NHS-activated sepharose), latex, cellulose, and ferro- or ferrimagnetic particles.

The ligand may be coupled to the solid support either covalently or non-covalently. Non-covalent binding includes binding via biotin affinity ligands binding to steptavidin matrices. Antibodies may be coupled non-covalently to protein A or protein G containing supports.

Preferably, the ligand is covalently coupled to the solid support.

Methods for immobilizing compounds on solid supports are known in the art. In general, before the coupling, the matrixes can contain active groups such as NHS, Carbodimide etc. to enable the coupling reaction with the ligand. The ligand can be coupled to the solid support by direct coupling (e.g. using functional groups such as amino-, sulfhydryl-, carboxyl-, hydroxyl-, aldehyde-, and ketone groups) and by indirect coupling (e.g. via biotin, biotin being covalently attached to the ligand and non-covalent binding of biotin to streptavidin which is bound directly to the solid support). The linkage to the solid support material may involve cleavable and non-cleavable linkers. The cleavage may be achieved by enzymatic cleavage or treatment with suitable chemical methods. The linker may be a C₁₋₁₀ alkylene group, which is optionally interrupted or terminated by one or more atoms or functional groups selected from the group consisting of S, O, NH, C(O)O, C(O), and C(O)NH and wherein the linker is optionally substituted with one or more substituents independently selected from the group consisting of halogen, OH, NH₂, C(O)H, C(O)NH₂, SO₃H, NO₂, and CN. The term “C₁₋₁₀ alkylene” means an alkylene chain having 1-10 carbon atoms, e.g. methylene, ethylene, —CH═CH—, —C≡C—, n-propylene and the like, wherein each hydrogen of a carbon atom may be replaced by a substituent.

According to a further step of the present invention, the protein preparation is contacted with the immobilized ligand under conditions allowing the formation of a complex between the immobilized ligand and the target.

In the present invention, the term “a complex between the immobilized ligand and the target” denotes a complex where the immobilized ligand interacts with the target, e.g. by covalent or, most preferred, by non-covalent binding.

In the context of the present invention, the term “under conditions allowing the formation of the complex” includes all conditions under which such formation, preferably such binding is possible. This includes the possibility of having the solid support on an immobilized phase and pouring the lysate onto it. In another preferred embodiment, it is also included that the solid support is in a particulate form and mixed with the cell lysate. Such conditions are known to the person skilled in the art.

In the context of non-covalent binding, the binding between the immobilized ligand and the target is, e.g., via salt bridges, hydrogen bonds, hydrophobic interactions or a combination thereof.

In a preferred embodiment, the steps of the formation of said complex are performed under essentially physiological conditions. The physical state of proteins within cells is described in Petty, 1998 (Howard R. Petty, Chapter 1, Unit 1.5 in: Juan S. Bonifacino, Mary Dasso, Joe B. Harford, Jennifer Lippincott-Schwartz, and Kenneth M. Yamada (eds.) Current Protocols in Cell Biology Copyright © 2003 John Wiley & Sons, Inc. All rights reserved. DOI: 10.1002/0471143030.cb0101s00Online Posting Date: May, 2001Print Publication Date: October, 1998).

The contacting under essentially physiological conditions has the advantage that the interactions between the ligand, the cell preparation (i.e. the phosphatidylinositol kinase to be characterized) and optionally the compound reflect as much as possible the natural conditions. “Essentially physiological conditions” are inter alia those conditions which are present in the original, unprocessed sample material. They include the physiological protein concentration, pH, salt concentration, buffer capacity and post-translational modifications of the proteins involved. The term “essentially physiological conditions” does not require conditions identical to those in the original living organism, wherefrom the sample is derived, but essentially cell-like conditions or conditions close to cellular conditions. The person skilled in the art will, of course, realize that certain constraints may arise due to the experimental set-up which will eventually lead to less cell-like conditions. For example, the eventually necessary disruption of cell walls or cell membranes when taking and processing a sample from a living organism may require conditions which are to not identical to the physiological conditions found in the organism. Suitable variations of physiological conditions for practicing the methods of the invention will be apparent to those skilled in the art and are encompassed by the term “essentially physiological conditions” as used herein. In summary, it is to be understood that the term “essentially physiological conditions” relates to conditions close to physiological conditions, as e.g. found in natural cells, but does not necessarily require that these conditions are identical.

For example, “essentially physiological conditions” may comprise 50-200 mM NaCl or KCl, pH 6.5-8.5, 20-37° C., and 0.001-10 mM divalent cation (e.g. Mg++, Ca++,); more preferably about 150 m NaCl or KCl, pH7.2 to 7.6, 5 mM divalent cation and often include 0.01-1.0 percent non-specific protein (e.g. BSA). A non-ionic detergent (Tween, NP-40, Triton-X100) can often be present, usually at about 0.001 to 2%, typically 0.05-0.2% (volume/volume). For general guidance, the following buffered aequous conditions may be applicable: 10-250 mM NaCl, 5-50 mM Tris HCl, pH5-8, with optional addition of divalent cation(s) and/or metal chelators and/or non-ionic detergents.

Preferably, “essentially physiological conditions” mean a pH of from 6.5 to 7.5, preferably from 7.0 to 7.5, and/or a buffer concentration of from 10 to 50 mM, preferably from 25 to 50 mM, and/or a concentration of monovalent salts (e.g. Na or K) of from 120 to 170 mM, preferably 150 mM. Divalent salts (e.g. Mg or Ca) may further be present at a concentration of from 1 to 5 mM, preferably 1 to 2 mM, wherein more preferably the buffer is selected from the group consisting of Tris-HCl or HEPES.

The skilled person will appreciate that between the individual steps of the methods of the invention, washing steps may be necessary. Such washing is part of the knowledge of the person skilled in the art. The washing serves to remove non-bound components of the cell lysate from the solid support. Nonspecific (e.g. simple ionic) binding interactions can be minimized by adding low levels of detergent or by moderate adjustments to salt concentrations in the wash buffer.

In a further step of the method of the invention, the amount of complexes formed in the step above is determined. In general, the less complex in the presence of the respective compound is formed, the stronger the respective compound interacts with the target, which is indicative for its therapeutic potential.

The detection of the complex formed according to the invention can be performed by using labeled antibodies directed against the target and a suitable readout system.

In the course of the present invention, it is preferred that the target is separated from the immobilized ligand in order to determine the amount of said complex. After this separation, the amount of the target may be determined.

According to invention, separating means every action which destroys the interactions between the immobilized ligand and the target. This includes in a preferred embodiment the elution of the target from the immobilized ligand.

The elution can be achieved by using non-specific reagents as described in detail below (ionic strength, pH value, detergents). In addition, it can be tested whether a compound of interest can specifically elute the target from the immobilized ligand.

Such non-specific methods for destroying the interaction are principally known in the art and depend on the nature of the ligand-target, preferably ligand-enzyme interaction. Principally, change of ionic strength, the pH value, the temperature or incubation with detergents are suitable methods to dissociate the target enzymes from the immobilized ligand. The application of an elution buffer can dissociate binding partners by extremes of pH value (high or low pH; e.g. lowering pH by using 0.1 M citrate, pH2-3), change of ionic strength (e.g. high salt concentration using NaI, KI, MgCl₂, or KCl), polarity reducing agents which disrupt hydrophobic interactions (e.g. dioxane or ethylene glycol), or denaturing agents (chaotropic salts or detergents such as Sodium-docedyl-sulfate, SDS; Review: Subramanian A., 2002, Immunoaffinty chromatography).

In some cases, the solid support has preferably to be separated from the released material. The individual methods for this depend on the nature of the solid support and are known in the art. If the support material is contained within a column the released material can be collected as column flowthrough. In case the support material is mixed with the lysate components (so called batch procedure) an additional separation step such as gentle centrifugation may be necessary and the released material is collected as supernatant. Alternatively magnetic beads can be used as solid support so that the beads can be eliminated from the sample by using a magnetic device.

Methods for the detection of the amount of a separated target are known in the art and include physico-chemical methods such as protein sequencing (e.g. Edmann degradation), analysis by mass spectrometry methods or immunodetection methods employing antibodies directed against the target.

Preferably, the amount of a target is determined by mass spectrometry or immunodetection methods.

Throughout the invention, if an antibody is used in order to detect the amount of a target (e.g. via ELISA), the skilled person will understand that, if a specific target is to be detected or if the amount of a target is to be determined, a specific antibody may be used (Sasaki et al., 2000, Nature 406, 897-902; Deora et al., 1998, J. Biol. Chem. 273, 29923-29928). As indicated above, such antibodies are known in the art. Furthermore, the skilled person is aware of methods for producing the same.

Suitable antibody-based assays include but are not limited to Western blots, ELISA assays, sandwich ELISA assays and antibody arrays or a combination thereof. The establishment of such assays is known in the art (Chapter 11, Immunology, pages 11-1 to 11-30 in: Short Protocols in Molecular Biology. Fourth Edition, Edited by F. M. Ausubel et al., Wiley, New York, 1999).

These assays can not only be configured in a way to detect and quantify a target (e.g. a catalytic or regulatory subunit of a kinase complex), but also to analyse posttranslational modification patterns such as phosphorylation or ubiquitin modification.

The identification of proteins with mass spectrometric analysis (mass spectrometry) is known in the art (Shevchenko et al., 1996. Analytical Chemistry 68: 850-858; Mann et al., 2001. Annual Review of Biochemistry 70, 437-473) and is further illustrated in the example section.

Preferably, the mass spectrometry analysis is performed in a quantitative manner, for example by stable isotope labelling, to create a specific mass tag that can be recognized by a mass spectrometer and at the same time provide the basis for quantification. These mass tags can be introduced into proteins or peptides metabolically, by chemical means, enzymatically, or provided by spiked synthetic peptide standards (Bantscheff et al., 2007; Anal. Bioanal. Chem. 389(4): 1017-1031).

Preferably, the mass spectrometry analysis is performed in a quantitative manner, for example by using iTRAQ technology (isobaric tags for relative and absolute quatification) or cICAT (cleavable isotope-coded affinity tags) (Wu et al., 2006. J. Proteome Res. 5, 651-658).

Alternatively, TMT isobaric tagging reagent can be used. The TMT reagents are a set of multiplexed, amine-specific, stable isotope reagents that can label peptides in up to six different biological samples enabling simultaneous identification and quantitation of peptides. The samples are analyzed with a nano-flow liquid chromatography system coupled online to a tandem mass spectrometer (LC-MS/MS) experiment followed by reporter ion quantitation in the MS/MS spectra (Ross et al., 2004. Mol. Cell. Proteomics 3(12):1154-1169; Dayon et al., 2008. Anal. Chem. 80(8):2921-2931; Thompson et al., 2003. Anal. Chem. 75(8):1895-1904).

According to a further preferred embodiment of the present invention, in case that the target is a protein, the characterization by mass spectrometry (MS) is performed by the identification of proteotypic peptides of the target. The idea is that the target is digested with proteases and the resulting peptides are determined by MS. As a result, peptide frequencies for peptides from the same source protein differ by a great degree, the most frequently observed peptides that “typically” contribute to the identification of this protein being termed “proteotypic peptide”. Therefore, a proteotypic peptide as used in the present invention is an experimentally well observable peptide that uniquely identifies a specific protein or protein isoform.

According to a preferred embodiment, the characterization is performed by comparing the proteotypic peptides obtained in the course of practicing the methods of the invention with known proteotypic peptides. Since, when using fragments prepared by protease digestion for the identification of a protein in MS, usually the same proteotypic peptides are observed for a given target, it is possible to compare the proteotypic peptides obtained for a given sample with the proteotypic peptides already known for said target and thereby identifying the target being present in the sample.

The result of these steps of the invention is how many complexes between the immobilized ligand and the target have been formed (ligand-complexes). Since the compound has been taken up by the cells of the animal before the target has been brought into contact with the immobilized ligand, the less ligand-complexes have been formed, the more targets have been bound by the compound. Consequently, the less ligand-complexes have been formed, the higher is the occupancy of the target with the compound, indicating a high compound-target interaction in vivo.

In a further step of the method of the present invention, the amount of complexes is correlated with the concentration of the compound in the body fluid sample.

This step is important since it is the purpose of the present invention to evaluate the target-compound interaction in vivo. If the concentration of the compound in the body fluid is low despite of a high amount of complexes, this would indicate that the compound is potentially very effective in binding the target.

In a preferred embodiment of the invention, further the pharmacological effect of the compound administered to the animal is determined. This effect will depend on nature of the compound and of the physiological role of the target. Preferably, said pharmacological effect is an anti-inflammatory or an anti-proliferative effect. Methods for determining such pharmacological effects are known in the art. For example, a drug intended for the treatment of autoimminue diseases can be tested in a keyhole limpet hemocyanin (KLH)-delayed-type hypersensitivity (DTH) rodent model (Engstrom et al. 2009. Internat. Immunopharmacol. 9, 1218-1227).

In a further preferred embodiment, the degree of the pharmacological effect is correlated with the amount of complexes and/or with the concentration of the compound. This correlation will provide an even better evaluation of the compound-target interaction.

Preferably, the effects of the compound are compared to the effects observed in an animal administered with a control compound or in an untreated animal. In this context, the term effect means the concentration of the compound in the biological fluid, the amount of complexes formed between the immobilized ligand and the target as well as the pharmacological effect of the compound. Consequently, in this preferred embodiment of the present invention, the effects of a compound are normalized by comparing them to the effects observed in an animal which has been administered with a control vehicle or which has not been treated.

In a preferred embodiment, the amount of complexes formed in protein preparations derived from different cellular samples is determined. By this embodiment, it is possible to compare the effects of the compound in different tissues, organs or samples. Furthermore, according to another preferred embodiment of the invention, it is also possible that the compound is evaluated in at least two animals derived from different species.

The present invention may also be used to test the effects of compounds which do not interact directly with the target but which are metabolized in the body and then the metabolite interacts with the target. Consequently, in a preferred embodiment, the compound is metabolized in the animal and the metabolite interacts with the target.

The present invention enables also to determine dose response curves for a given compound. Consequently, in a preferred embodiment, by correlating the amount of complexes with the concentration of the compound in the body fluid sample a dose-response curve is obtained.

In a further preferred embodiment, at least two body fluid samples and at least two cellular samples are taken at different time points in order to measure the time dependence of the complex formation. This is important if the compound-target interaction over the time should be observed and correlated with the onset, duration and decline of the pharmacological effect. This embodiment is especially useful for characterizing irreversible inhibitors as explained above.

The compound evaluated by the method of the present invention may be used as a drug in medical treatment.

The invention is further illustrated by the following figures and examples, which are not considered as being limiting for the scope of protection conferred by the claims of the present application.

### BRIEF DESCRIPTION OF THE FIGURES

FIG. 1: Structure of compound 1

FIG. 2: Pharmacokinetic analysis of compound 1. Single doses of 30 mg/kg and 100 mg/kg were administered orally to rats. Plasma concentration of compound 1 was plotted against sampling time points. Each time point consists of the average (mean value±SD) of data from three rats.

FIG. 3: Relative target engagement (TE) of mTOR in rat organs for different administered doses (0, 30 or 100 mg/kg) of compound 1. Organs were collected after 8 hours of treatment.

FIG. 4: Relative target engagement (TE) of mTOR in rat organs plotted against measured concentration of compound 1 in plasma (8 hour time point).

### EXAMPLE

### Target Engagement Study 1

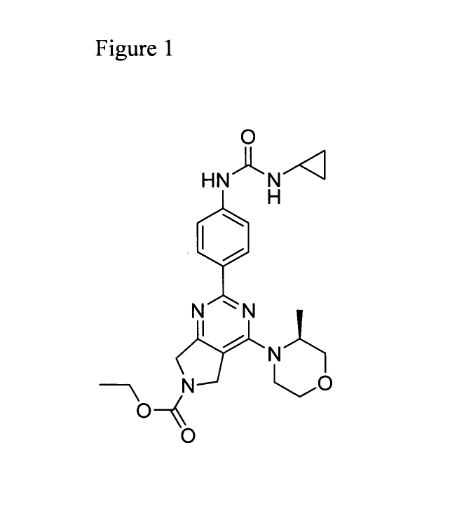

This example demonstrates the interaction of compound 1 (FIG. 1) with the mTOR kinase as target in vivo in three different rat organs (brain, kidney and liver). The synthesis of compound 1 is described in WO 2010/103094.

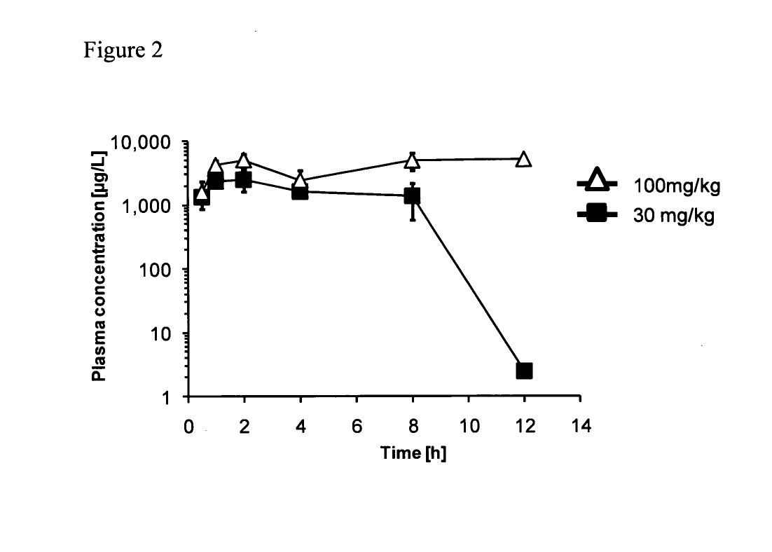

In a pharmacokinetic (PK) study male Wistar rats were dosed orally (p.o.) with a single dose of compound 1 at different doses (0, 30 or 100 mg/kg) as described in Table 1. Two control animals did not receive compound 1. After defined time points blood samples were taken according to the schedule in Table 2 and the compound concentration in the plasma samples was measured (FIG. 2).

After 8 hours animals 4 to 6 and 10 to 12 were sacrificed and organs (brain, kidney, liver) were collected and frozen at −80° C. for target engagement measurements. Lysates were prepared from the organs of the two control rats and two animals of the treatment groups and subsequently analysed with the kinobeads method as described (WO 2009/098021A1). The added affinity matrix (immobilized ligand) can only capture free mTOR that is not interacting with compound 1. Consequently, increasing occupancy of mTOR by compound 1 will lead to a reduction of captured mTOR protein (TE=1 means that the target is not occupied by compound 1 as in non-treated animals).

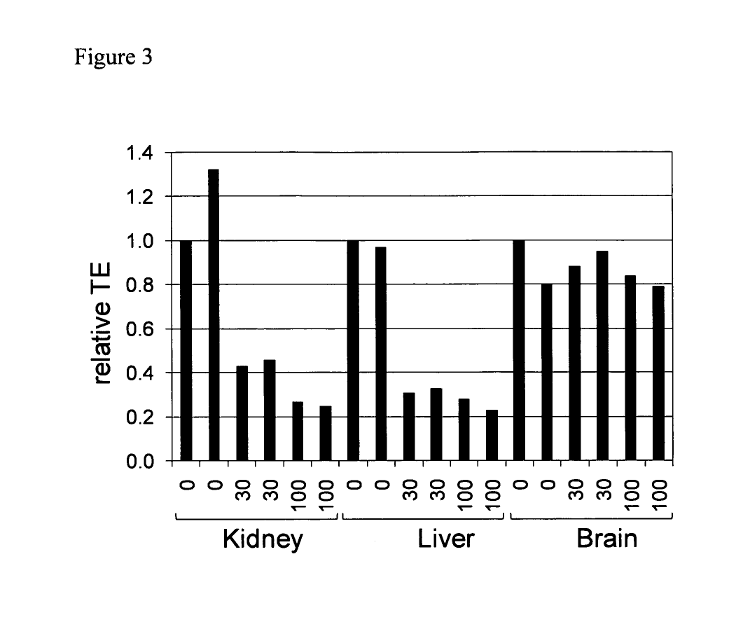

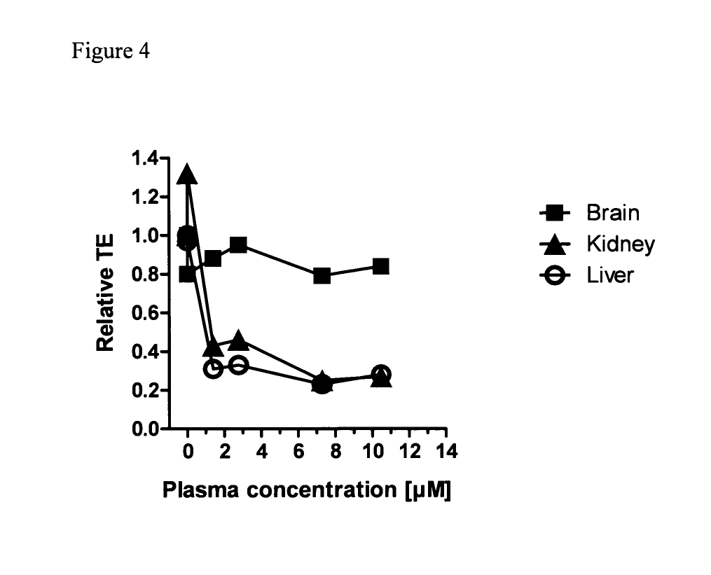

The results of the target engagement study are summarized in Table 4. FIG. 3 shows the relative target engagement (TE) values plotted against the administered dose of compound 1 (0, 30 and 100 mg/kg). For each organ the target engagement of mTOR in control animal 1 was set to 1.0 (target not occupied with drug) and the values for the other animals expressed in relation to this value. In kidney and liver there is a dose dependent decrease in captured mTOR indicating an increasing target occupancy. No such effect is seen in brain, presumably because compound 1 does not cross the blood brain barrier. In FIG. 4 the relative target engagement (TE) is correlated with the concentration of the compound as measured in plasma.

### 1. Animals and Pharmacokinetic Protocols

Male Wistar rats were obtained from Janvier (Le Genest St Isle, France). Animals were fasted 12 to 14 hours before dosing with compound and 2 hours post-dosing.

Compound 1 was administered orally as a single dose at 30 or 100 mg/kg (vehicle: 0.5% carboxymethylcellulose (CMC); application volume 5 ml/kg).

Blood was collected at time points indicated in Table 2. 300 μl of heparin blood per time point yielded 100 to 150 μl heparin plasma.

TABLE 1
Animal groups

| | | Dose | | Animals | Animal |
|---|---|---|---|---|---|
| Group | Compound | (mg/kg) | Route | (n) | number |
| 0 | — | 0 | po | 2 | control 1, |
| | | | | | control 2 |
| 1 | compound 1 | 30 | po | 6 | 1-6 |
| 2 | compound 1 | 100 | po | 6 | 7-12 |

TABLE 2
Animals, blood samples and time points

| | | | | | Bleed | |
|---|---|---|---|---|---|---|
| | | Dose | Animals | Animal | Time Points | Sample |
| Group | Compound | mg/kg | (n) | number | (minutes) | number |
| 1 | compound 1 | 30 | 3 | 1-3 | 0 (predose), | 1-6, 10-12, 19-21 |
| | | | | | 30, 120, 720 | |
| 1 | compound 1 | 30 | 3 | 4-6 | 60, 240, 480 | 7-9, 13-18 |
| 2 | compound 1 | 100 | 3 | 7-9 | 0 (predose), | 22-27, 31-33, 40-42 |
| | | | | | 30, 120, 720 | |
| 2 | compound 1 | 100 | 3 | 10-12 | 60, 240, 480 | 28-30, 34-39 |

### Analytical Protocols

Plasma concentrations of compound 1 were determined from rat plasma using a HPLC-MS/MS method. Reference items: Compound 1, batch 3. Wistar rat plasma, anti-coagulated with Li-heparinate.

### HPLC Equipment

### Columns: 100*2.1 mm, MZ-Analysentechnik, Mainz, Germany

### Pump: Agilent Series 1100, Chromtech, Idstein, Germany

### Degasser: Agilent Series 1100, Chromtech, Idstein, Germany

### Autosampler: CTC PAL, CTC Analytics, Zwingen, Switzerland

### Detector: API 2000, Applied Biosystems, Darmstadt, Germany

Computer: IBM compatible, Dell GmbH, Frankfurt, Germany

### Software: Analyst Software Vers. 1.2, Applied Biosystems, Darmstadt, Germany

### Sample Preparation of Compound 1

For linearity and quality control samples:
100 μl of rat plasma were used
10 μl standard formulation of compound 1 was added
shaking for approx 30 sec. on the laboratory shaker
addition of 300 μl acetonitrile
centrifugation for 5 min at 12000 rpm
transfer of 350 μl supernatant into a 1.0 ml tube
evaporation to dryness by rotational evaporation
reconstitution in 150 μl mobile phase A and B in a ratio of 70:30
centrifugation for 5 min at 12000 rpm
transfer of the clear supernatant into a sample vial

For study samples:
50 μl of rat plasma were used
5 μl water were added
shaking for approx 30 sec. on the laboratory shaker
addition of 150 μl acetonitrile
centrifugation for 5 min at 12000 rpm
transfer of 175 μl supernatant into a 1.0 ml tube
evaporation to dryness by rotational evaporation
reconstitution in 150 μl mobile phase A and B in a 70:30 ratio
centrifugation for 5 min at 12000 rpm
transfer of the clear supernatant into a sample vial

For all samples:
inject 30 μA into the HPLC system with a 20 μl sample loop.

HPLC Conditions for compound 1

Mobile Phase A: 0.02 M CH₃COONH₄, pH not adjusted plus 0.1% formic acid.
Mobile Phase B: 0.02 M CH₃COONH₄ in water, methanol acetonitrile (5:5:90) plus 0.1% formic acid pH not adjusted.
Flow rate: 0.25 ml/min
Column temperature: 30° C.
Gradient conditions:

| Time | A | B | Flow |
|---|---|---|---|
| [min] | [%] | [%] | [ml/min] |
| 0.0 | 70 | 30 | 0.25 |
| 1.0 | 70 | 30 | 0.25 |
| 6.0 | 10 | 90 | 0.25 |
| 10.0 | 10 | 90 | 0.25 |
| 10.5 | 70 | 30 | 0.25 |
| 20.0 | 70 | 30 | 0.25 |

### Detector: MS/MS

### Ionization Interface: Electro-spray

Ionisation mode: Positive

### Ion Voltage: 5.5 kV

Desolvation temperature: 400° C.

### Acquisition Method Compound 1_MRM_V1_{—}38 mm.dam

| Mass | | | Delustering | Collision | Dwell |
|---|---|---|---|---|---|
| transitions | Precursor | Product | potential | energy | time |
| (MRM) | ion | ion | (V) | (V) | (ms) |
| compound 1 | 467.4 | 410.2 | 70 | 40 | 150 |

### Data Processing

Pharmacokinetic analysis was performed with Pharsight WinNonlin Vers. 5.2.1 (St. Louis, Mo., USA) and statistical calculations with Microsoft Excel 2000 (Unterschleissheim, Germany).

2. Preparation of Cell Lysates from Organs

Organs were homogenized by mechanical disruption with a homogenizer (Polytron PT3100; Kinematica, Littau/Lucerne, Switzerland) by applying three 10 second pulses at 4° C. in lysis buffer (50 mM Tris-HCl, 5% glycerol, 150 mM NaCl, 1.5 mM MgCl₂, 25 mM NaF, 1 mM sodium vanadate, 1 mM DTT, pH 7.5; one complete EDTA-free tablet (protease inhibitor cocktail, Roche Diagnostics, 1 873 580) per 25 ml buffer was added). Then NP40 detergent was added to a final concentration of 0.8% and the suspension was transferred to a precooled dounce tissue grinder (Wheaton Science International, Millville, N.J., USA). The suspencion was dounced 10 times using a mechanized POTTER S homogenizer (B. Braun International, Göttingen, Germany). The homogenate was transferred to a 50 ml precooled falcon tube, incubated on ice for 30 minutes and centrifuged for 10 minutes at 6.000 g at 4° C. The supernatant was transferred to an ultracentrifuge polycarbonate tube (Beckman Coulter, Brea, Calif., USA; catalogue number 355654) and spun for one hour at 100.000 g at 4° C. The supernatant was transferred to a 50 ml Falcon tube and stored on ice. The protein concentration was determined by a Bradford assay (BioRad, Hercules, Calif., USA) and aliquots were quickly frozen in liquid nitrogen and stored at −80° C.

3. Capturing of Proteins from Cell Lysate

Sepharose-beads with the immobilized compound (35 μl beads per pull-down experiment) were equilibrated in lysis buffer and incubated with a cell lysate sample containing 5 mg of protein on an end-over-end shaker (Roto Shake Genie, Scientific Industries Inc.) for 2 hours at 4° C. Beads were collected, transferred to Mobicol-columns (MoBiTech 10055) and washed with 10 ml lysis buffer containing 0.4% NP40 detergent, followed by 5 ml lysis buffer containing 0.2% detergent. To elute bound proteins, 60 μl 2×SDS sample buffer was added to the column. The column was incubated for 30 minutes at 4° C. and the eluate was transferred to a siliconized microfuge tube by centrifugation. Proteins were then alkylated with 108 mM iodoacetamid. Proteins were then separated by SDS-Polyacrylamide electrophoresis (SDS-PAGE).

### 4. Protein Identification and Quantitation by Mass Spectrometry

The cell lysate was contacted with the affinity matrix (immobilized ligand) to capture free mTOR protein. The proteins bound to the immobilized compound were eluted with detergent-containing buffer, separated on a SDS-polyacryamide gel and analyzed by mass spectrometry. The peptide extracts corresponding to animals treated with different doses of compound 1 were treated with different variants of the isobaric tagging reagent (TMT-reagents, Thermofisher). The TMT reagents are a set of multiplexed, amine-specific, stable isotope reagents that can label peptides in up to six different biological samples enabling simultaneous identification and quantitation of peptides. The TMT reagents were used according to instructions provided by the manufacturer. The combined samples were analyzed with a nano-flow liquid chromatography system coupled online to a tandem mass spectrometer (LC-MS/MS) experiment followed by reporter ion quantitation in the MS/MS spectra (Ross et al., 2004. Mol. Cell. Proteomics 3(12):1154-1169; Dayon et al., 2008. Anal. Chem. 80(8):2921-2931; Thompson et al., 2003. Anal. Chem. 75(8):1895-1904). Further experimental protocols can be found in WO2006/134056 and a previous publication (Bantscheff et al., 2007. Nature Biotechnology 25, 1035-1044).

### 4.1 Protein Digestion Prior to Mass Spectrometric Analysis

Gel-separated proteins were digested in-gel essentially following a previously described procedure (Shevchenko et al., 1996, Anal. Chem. 68:850-858). Briefly, gel-separated proteins were excised from the gel using a clean scalpel, destained twice using 100 μl 5 mM triethylammonium bicarbonate buffer (TEAB; Sigma T7408) and 40% ethanol in water and dehydrated with absolute ethanol. Proteins were subsequently digested in-gel with porcine trypsin (Promega) at a protease concentration of 10 ng/μl in 5 mM TEAB. Digestion was allowed to proceed for 4 hours at 37° C. and the reaction was subsequently stopped using 5 μl 5% formic acid.

### 4.2 Sample Preparation Prior to Analysis by Mass Spectrometry

Gel plugs were extracted twice with 20 μl 1% formic acid and three times with increasing concentrations of acetonitrile. Extracts were subsequently pooled with acidified digest supernatants and dried in a vacuum centrifuge.

### 4.3 TMT Labeling of Peptide Extracts

The peptide extracts of samples corresponding to organs of rats dosed with different concentrations of compound 1 were treated with different variants of the isobaric tagging reagent (TMT sixplex Label Reagent Set, part number 90066, Thermo Fisher Scientific Inc., Rockford, Ill. 61105 USA). The TMT reagents are a set of multiplexed, amine-specific, stable isotope reagents that can label peptides on amino groups in up to six different biological samples enabling simultaneous identification and quantitation of peptides. The TMT reagents were used according to instructions provided by the manufacturer. The samples were resuspended in 10 μl 50 mM TEAB solution, pH 8.5 and 10 μl acetonitril were added. The TMT reagent was dissolved in acetonitrile to a final concentration of 24 mM and 10 μl of reagent solution were added to the sample. The labeling reaction was performed at room temperature for one hour on a horizontal shaker and stopped by adding 5 μl of 100 mM TEAB and 100 mM glycine in water. The labelled samples were then combined, dried in a vacuum centrifuge and resuspended in 60% 200 mM TEAB/40% acetonitril. 2 μl of a 2.5% NH2OH solution in water were added, incubated for 15 min and finally the reaction was stopped by addition of 10 μl of 20% formic acid in water. After freeze-drying samples were resuspended in 50 μl 0.1% formic acid in water.

### 4.4 Mass Spectrometric Data Acquisition

Peptide samples were injected into a nano LC system (CapLC, Waters or nano-LC 1D+, Eksigent) which was directly coupled either to a quadrupole TOF (QTOF Ultima, QTOF Micro, Waters), ion trap (LTQ) or Orbitrap mass spectrometer. Peptides were separated on the LC system using a gradient of aqueous and organic solvents (see below). Solvent A was 0.1% formic acid and solvent B was 70% acetonitrile in 0.1% formic acid.

TABLE 3
Peptides elution off the LC system

| | | Gradient |
|---|---|---|
| | Flow rate | Time (min)- |
| Method file | (nL/min) | % B |
| PQD_265min | 190 | 00-5.263 |
| | | 07-10 |
| | | 190-40.263 |
| | | 210-52.105 |
| | | 223-60 |
| | | 230-90 |
| | | 236-90 |
| | | 240-5.263 |
| | | 260-5.263 |

### 4.5 Protein Identification and Quantitation

The peptide mass and fragmentation data generated in the LC-MS/MS experiments were used to query a protein data base consisting of an in-house curated version of the International Protein Index (IPI) protein sequence database combined with a decoy version of this database (Elias and Gygi, 2007, Target-decoy search strategy for increased confidence in large-scale protein identifications by mass spectrometry. Nature Methods 4, 207-214). Proteins were identified by correlating the measured peptide mass and fragmentation data with data computed from the entries in the database using the software tool Mascot (Matrix Science; Perkins et al., 1999. Probability-based protein identification by searching sequence databases using mass spectrometry data. Electrophoresis 20, 3551-3567). Search criteria varied depending on which mass spectrometer was used for the analysis. Protein acceptance thresholds were adjusted to achieve a false discovery rate of below 1% as suggested by hit rates on the decoy data base (Elias and Gygi, 2007, Target-decoy search strategy for increased confidence in large-scale protein identifications by mass spectrometry. Nature Methods 4, 207-214).

Relative protein quantitation was performed using peak areas of iTMT reporter ion signals essentially as described in an earlier publication (Bantscheff et al., 2007. Nature Biotechnology 25, 1035-1044).

TABLE 4
Compound concentration of compound 1 in plasma (exposure) and relative target
engagement (TE) of mTOR in rat organs

| | | | | | Conc. | Conc. | | | |
|---|---|---|---|---|---|---|---|---|---|
| | | | | | in | in | | | |
| Dose | | | Sample | Time | plasma | plasma | TE | TE | TE |
| mg/kg | Group | Animal | number | (hours) | μg/L | μM | Kidney | Brain | Liver |
| 0 | 0 | Control 1 | — | — | — | — | 1.00 | 1.00 | 1.00 |
| 0 | 0 | Control 2 | — | — | — | — | 1.32 | 0.80 | 0.97 |
| 30 | 1 | 4 | 16 | 8 | 656 | 1.41 | 0.43 | 0.88 | 0.31 |
| 30 | 1 | 5 | 17 | 8 | 1290 | 2.77 | 0.46 | 0.95 | 0.33 |
| 100 | 2 | 11 | 38 | 8 | 3400 | 7.29 | 0.25 | 0.79 | 0.23 |
| 100 | 2 | 10 | 37 | 8 | 4890 | 10.48 | 0.27 | 0.84 | 0.28 |

1. A method for evaluation of a compound-target interaction in vivo, comprising the steps of
a) administering the compound to an animal,
b) taking a body fluid sample of the animal,
c) determining the concentration of the compound in the body fluid sample,
d) taking a cellular sample of the animal containing the target,
e) preparing a protein preparation of said cellular sample,
f) providing an immobilised ligand capable of binding to the target,
g) contacting the protein preparation with the immobilised ligand under conditions allowing the formation of a complex between the immobilised ligand and the target,
h) determining the amount of complexes formed in step g), and
i) correlating the amount of complexes with the concentration of the compound in the body fluid sample.

2. The method of claim 1, wherein further the pharmacological effect of the administration of the compound to the animal is determined.

3. The method of claim 2, wherein the degree of the pharmacological effect is correlated with the amount of complexes and/or with the concentration of the compound.

4. The method of claim 1, wherein the effects of the compound are compared to the effects observed in an animal administered with a control compound.

5. The method of claim 1, wherein the target is a protein, preferably an enzyme which is preferably a kinase.

6. The method of claim 1, wherein the compound is a modulator, preferably an inhibitor of the target.

7. The method of claim 1, wherein the body fluid sample is derived from blood, plasma, lymph liquid, or cerebrospinal fluid of the animal.

8. The method of claim 1, wherein the cellular sample is derived from the blood, an organ or a tumor tissue of the animal.

9. The method of claim 1, wherein the amount of complexes formed in protein preparations derived from different cellular samples is determined.

10. The method of claim 1, wherein the compound is metabolized in the animal and the metabolite interacts with the target.

11. The method of claim 1, wherein the compound is evaluated in at least two animals derived from different species.

12. The method of claim 1, wherein by correlating the amount of complexes with the concentration of the compound in the body fluid sample a dose-response curve is obtained.

13. The method of claim 1, wherein at least two body fluid samples and at least two cellular samples are taken at different time points in order to measure the time dependence of the complex formation.

14. The method of claim 1, wherein the pharmacological effect is an anti-inflammatory or an anti-proliferative effect.

15. The method of claim 1, wherein the compound is an antibody.

16. The method of claim 1, wherein the ligand is an antibody.
